(19) United States
(12) Patent Application Publication
(10) Pub. No.: US 2014/0163362 A1
(43) Pub. Date: Jun. 12, 2014

(54) CARDIAC MICROWAVE SIGNAL DETERMINATION OF CARDIOVASCULAR DISEASES

(71) Applicant: California Institute of Technology, Pasadena, CA (US)

(72) Inventors: Niema Pahlevan, Pasadena, CA (US); Robert R. McGrath, Pasadena, CA (US); Morteza Gharib, Altadena, CA (US)

(73) Assignee: CALIFORNIA INSTITUTE OF TECHNOLOGY, Pasadena, CA (US)

(21) Appl. No.: 13/956,177

(22) Filed: Jul. 31, 2013

Related U.S. Application Data
(60) Provisional application No. 61/738,229, filed on Dec. 17, 2012.
(60) Provisional application No. 61/678,425, filed on Aug. 1, 2012.

(51) Int. Cl. A61B 5/05; A61B 5/02; A61B 5/021

(52) U.S. Cl. CPC A61B 5/0507; A61B 5/021; A61B 5/02
USPC 600/430

(57) ABSTRACT

A microwave transceiver and feature extraction system is described. This system is adapted for measuring both electrical (ECG-related waveforms) and mechanical activity (heart sound and wall motion) of the heart and vessels, determining which signal features are related to which mechanical properties, and measurement of important hemodynamic parameters such as pressure, flow, and vessel's wall displacement. This system is non-invasive, portable, non-contacting and can remotely collect data at distances of <1 m to several meters that make it a perfect device for telemedicine.

### CROSS-REFERENCE TO RELATED APPLICATIONS

This application claims the benefit of and priority to U.S. Provisional Patent Application Ser. Nos. 61/678,425, filed Aug. 1, 2012, and 61/738,229, filed Dec. 17, 2012, each of which is incorporated by reference herein in its entirety for all purposes.

### FEDERAL SUPPORT

Inventive subject matter described herein was made in the performance of work under a NASA contract, and is subject to the provisions of Public Law 96-517 (35 USC 202) in which the Contractor has elected to retain title.

### FIELD

The subject matter described herein relates to remote (standoff), non-invasive, and non-contacting determination of cardiovascular diseases based on use of the cardiac microwave signal.

### BACKGROUND

Cardiovascular diseases (CVDs) are the underlying cause of about one of every three deaths in United States each year. Likewise, about 34% of American adults are suffering from one or more types of CVD. In 2010, the total direct and indirect cost of CVDs was approximately $503 billion.

Certainly, there is an urgent need to develop new methods and devices for diagnosing and monitoring CVDs. Diagnosis enables early intervention and remediation. Monitoring may be a useful tool in each of behavior modification and prediction/avoidance of an acute event leading to emergency hospitalization, morbidity and/or mortality. New methods and devices to meet these need(s) advantageously employ non-invasive measurement to reduce medical complications and increase patient comfort. Ideally, they are also easy to use by medical personnel and subjects in a home environment.

As disclosed in U.S. Pat. Nos. 7,272,431 and 7,811,234 (each incorporated herein by reference in its entirety for all purposes), it has been shown that a properly prepared microwave signal can be safely reflected off of a human, and the reflected signal has features that can be correlated with certain electrical and mechanical activities of the heart. This microwave signal is referred to further below as “the cardiac microwave” (CMW) signal. As disclosed in U.S. Pat. Nos. 7,889,053 and 8,232,866 (each patent also incorporated by reference in its entirety for all purposes) the CMW signal may also function as a long-standoff biometric. Additional improvements and applications of the CMW measurement technique for cardiac disease diagnosis are presented below meeting aforementioned public-health needs.

### SUMMARY

The present subject matter includes devices and systems (e.g., including the sensor hardware referenced herein and the addition of a computer processor and other ancillary/support electronics and various housing elements) and methods (including the hardware and software for carrying out the same) meeting some or all of aforementioned needs. Such methods and devices are adapted for analysis of the cardiac microwave signal (CMW) in a remote (standoff), non-invasive, and non-contacting fashion, remotely collecting data at distances of <1 m to several meters. Embodiments of a microwave transceiver and feature extraction system are described. This system is adapted for measuring both electrical (ECG-related waveforms) and mechanical activity (heart sound and wall motion) of the heart and vessels, determining which signal features are related to which mechanical properties, and measurement of hemodynamic parameters such as pressure, flow, and vessel wall displacement.

A CMW signal is related to the motion of tissues and organs such as the heart, aorta, and other compliant conduits and vessels in the body. There is a good correlation between the ICG (Impedance Cardiogram, which relates to volume change; ΔVαΔZ) and the CMW. In addition, data taken on a human leg shows that the shape of the first derivative of CMW is similar to the pressure wave of the femoral artery. Considering the fact that the pressure wave is almost the same as the wall displacement wave at central arteries (due to low degree of viscoelasticity), CMW therefore presents a strong correlation with arterial wall motion as well as other biological membranes and vessels.

A first example method involves the application of the CMW signal on detecting wall displacement of aorta and other compliant vessels and conduits in the body. In the case of the aorta and large central arteries, since the wall displacement wave has the same waveform as the blood pressure wave, this method can be used as a non-contacting, non-invasive technique for pressure measurement. Furthermore, by non-invasive determination of the central pressure, other important vascular indices such as compliance, pulse pressure, augmentation index, etc. can be determined non-invasively and remotely.

A second example method involves the application of the CMW on detecting the motion of the heart wall. A healthy heart has a specific wall motion in each phase of the cardiac cycle to ensure optimized function. However, under disease condition such as systolic heart failure, diastolic heart failure, dilated cardiomyopathy, etc. all or some of the motion phases do not follow the healthy heart's wall motion pattern. Therefore, a method and device based on the CMW measurement technique can be used for non-invasive, non-contacting (and even remote) diagnosis of heart diseases and/or valvular diseases.

### BRIEF DESCRIPTION OF THE DRAWINGS

The figures provided herein may be diagrammatic and not necessarily drawn to scale, with some components and features exaggerated and/or abstracted for clarity. The graphs provide are drawn to scale and may be relied upon for claim support. Variations from the embodiments pictured are contemplated. Accordingly, depiction of aspects and elements in the figures are not intended to limit the scope of the claims, except when such intent is explicitly stated—per above—or otherwise.

FIG. 1A is an overview of an example embodiment of the system.

FIG. 1B is an electronic hardware diagram of an example embodiment of a CMW system that may be incorporated in the embodiment of FIG. 1A.

FIGS. 2-4 are example graphs comparing CMW system performance against simultaneously-measured alternative biometric signals.

FIG. 5A is an example graph of a hemodynamic waveform and FIG. 5B is an example graph of CMW signal measurement and its derivative for comparison to the waveform in FIG. 5A.

### DETAILED DESCRIPTION

Various example embodiments are described below. Reference is made to these examples in a non-limiting sense. They are provided to illustrate more broadly applicable aspects of inventive aspects. Various changes may be made to the embodiments described and equivalents may be substituted without departing from their true spirit and scope. In addition, many modifications may be made to adapt a particular situation, material, composition of matter, process, process act(s) or step(s) to the objective(s), spirit or scope of the claims made herein.

In the subject methods and systems, microwave signals in the frequency range of 0.5 GHz to 100 GHz are used for non-invasive, non-contacting, and/or remote measurement. In doing so, the subject CMW technique may be used to sense, record, and/or monitor the wall motion of the heart. Alternatively, a determination of the wall motion of the heart can be made my measuring the corresponding micro-motions at the surface of the torso using the CMS technique. Likewise, the CMS technique may be used for determination of aortic wall motion and wall motion of other compliant vessels and conduits.

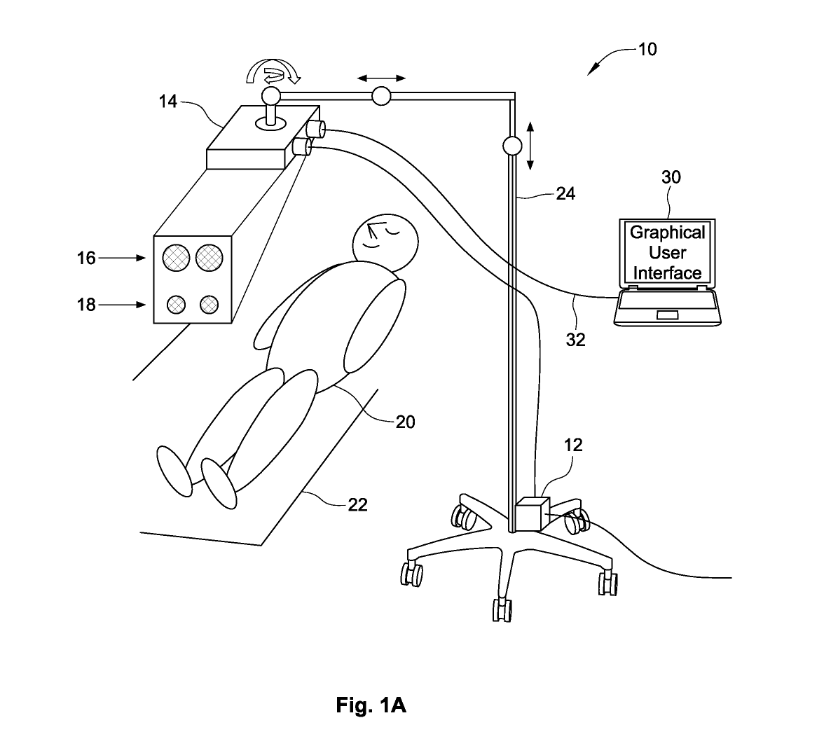

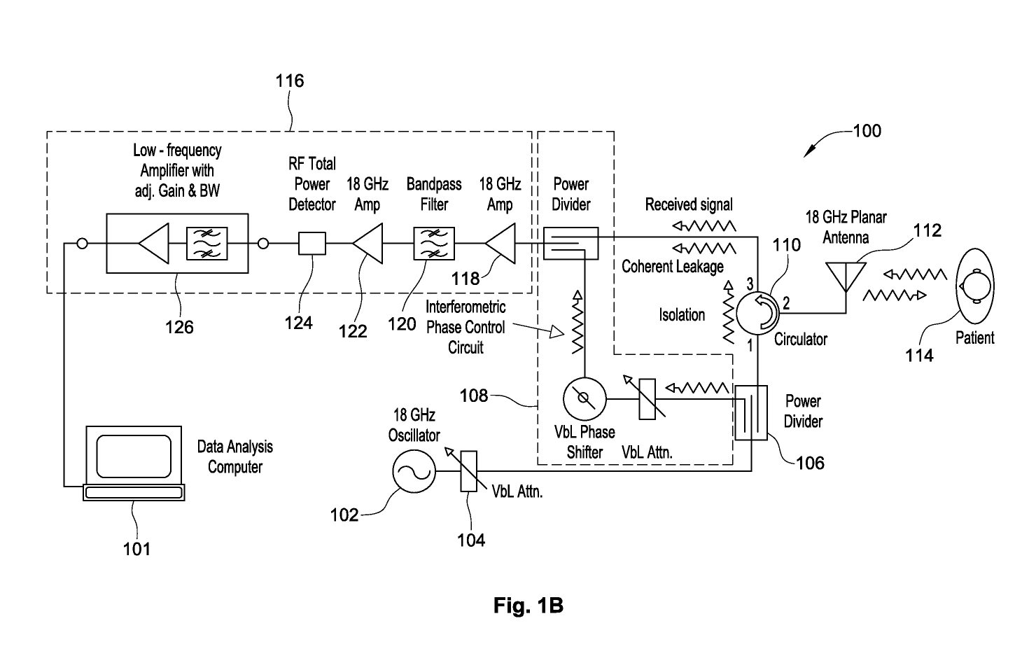

FIG. 1 illustrates an example embodiment of a CMW system 10. All electronic components (e.g., as in FIG. 1B) except the power supply 12 (for thermal reasons) may be housed in small (e.g., 20 cm×20 cm×5 cm) box 14. Here, planar antennas 16 (low frequency), 18 (high frequency) are located at/on a bottom face (illustrated by offset view) with a patient 20 lying on a bed or on a conventional medical examination table 22.

A two-frequency instrument with bi-static RF sub-systems is shown. Box 14 (and included components) is optionally light enough to mount on a simple adjustable stand 24 (similar to a conventional IV-fluids stand) for easy positioning over the patient or otherwise. For such a system, various angular/rotational, length and height adjustment options are indicated by arrows.

Analysis and control interface software may employ an intuitive Graphical User Interface (GUI) that can be supplied on a DVD for installation on the user's available desktop, laptop computer 30, or a dedicated console. The computer may connect to the instrument box 14 with a conventional USB cable 32 or wirelessly. The entire instrument can be designed to be collapsible or folded-up and fit into an easily transported carrying-case. Such an inexpensive and compact instrument for CVD diagnosis could have a major impact on the Medical community.

Suitable CMW circuitry is presented in the Background patents referenced above and as illustrated in FIG. 1B. System 100 includes a computer or signal processing system 101 and a number of other components forming a microwave cardiac measurement system. As illustrated, an 18 GHz oscillator 102 serves as the signal source. Power level is controlled by a 20 dB variable attenuator 104. The signal is then split by a 3 dB power divider 106. Half of the signal goes into a phase control circuit 108, and half goes to a circulator 110 where it is routed to a high-gain patch-array planar antenna 112. It is radiated in a narrow beam toward the patient or subject of interest 114 (the radiated power is typically in the range of about 50 microwatts to about 1 milliwatt). The signal reflected from this person is received by the same antenna 112, and routed by the circulator 110 to the receiver portion 116 of the system.

Since real world components are not perfect, some of the source signal leaks the wrong direction around the circulator 110 and is injected directly into the receiver portion 116 of the system. This is where the phase control circuit 108 is used. The signal power coupled into it is coherent with the leakage signal of the isolator port of the circulator 110. By adjusting phase and amplitude of the signal in the phase control circuit to compensate for the leakage signal then coupling this adjusted signal back into the receiver path, the overall phase sensitivity of the system can be controlled. The signal is then amplified by approximately 30 dB by a low-noise 18 GHz amplifier 118. In some embodiments, the phase control circuit 108 is also configured to reduce the effects of gross body motion. In one such embodiment, the phase control circuit is configured primarily to reduce the effects of gross body motion and secondarily to compensate for the leakage signal.

The signal in the receiver path is then filtered using a bandpass filter 120. The bandwidth of the filter can be in the range of about 18 MHz to 360 MHz. Bandpass filters 120 are used to reduce the overall noise of the receiver section to a desired level. The signal is then further amplified by about 30 dB using a second amplifier 122. A square-law, direct detector 124 can be used to measure the total power in the signal. The output of the detector 124 contains the low-frequency cardiac-related modulation of the 18 GHz signal power. This low-frequency signal is further amplified and filtered in block 126 to optimize the signal-to-noise ratio. The signal is then digitized and analyzed to retrieve information per the examples below. Such analysis may include determining a physiological condition and outputting a signal corresponding to the physiological condition.

### EXAMPLES

The following examples are provided by way of illustration of the above, demonstrating the correlations that may be employed in the subject diagnosis. In each case, a CMW signal generated for a test subject is comparable to another biometric measurement or set of measurements presently employed in patient monitoring and/or diagnosis. A continuous-wave (CW) microwave transceiver system was developed that is capable, for example, of accurately monitoring (+/−5%) the heart rate of a (cooperatively) moving subject (walking back or forth in the microwave beam). This system employed an “interferometeric type” of phase control loop to reduce RF leakage from the transmitter into the receiver channel (which is the primary source of gross motion artifacts) and learning algorithms to extract cardiac features.

### Example of Correlation Between ICG and a CMW Signal at 18 GHz

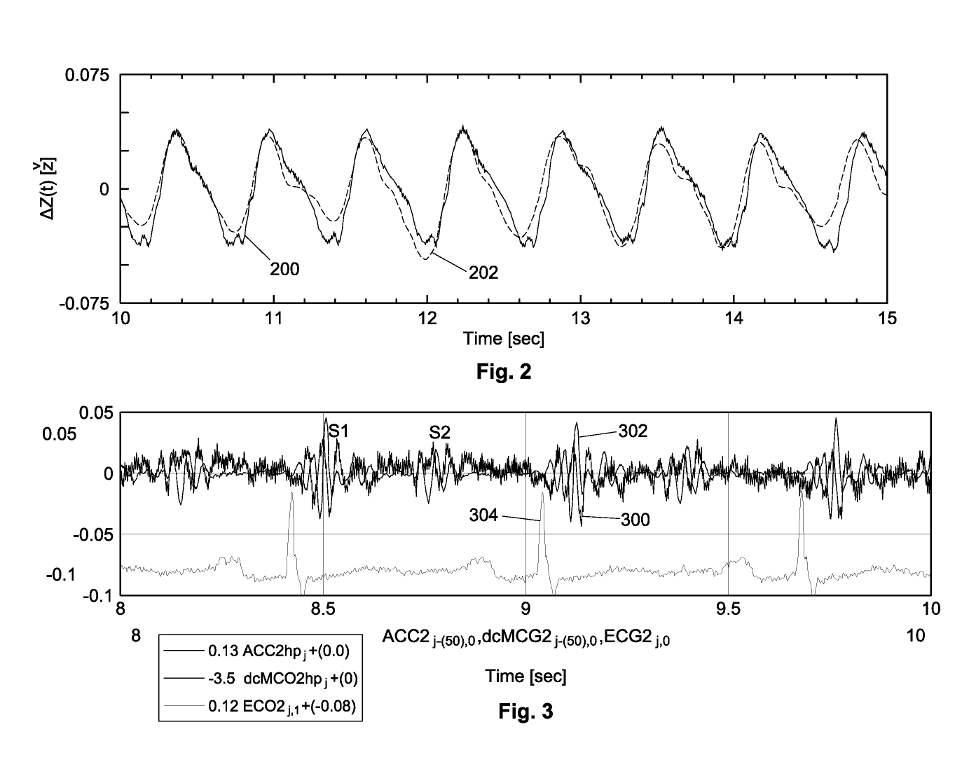

FIG. 2 shows CMW signal features (solid line 200) that correlate with a simultaneously-measured Impedance Cardiogram (ICG) signal (dashed line 202).

### Example of Correlation Between PCG and CMW Signal at 18 GHz

As illustrated in FIG. 3, features extracted from the CMW signal 300 correlate well with a simultaneously measured phonocardiogram (PCG) signal 302. Detailed features of the PCG can also be extracted from the CMW signal. As labeled, S1 is the First Heart Sound and S2 is the Second Heart Sound. Note also a (contact-measured) ECG signal 304 for reference.

### Example of Correlation Between ECG and CMW Signal at 2.5 GHz

FIG. 4 illustrates correlation between an ECG signal 400 and a CMW signal 402. Features in the CMW signal correlate well with P- and T-waves of the ECG. The various features that can be matched in each signal are shown labeled in the figure.

### Example of Correlation Between Pressure Wave and CMW Signal

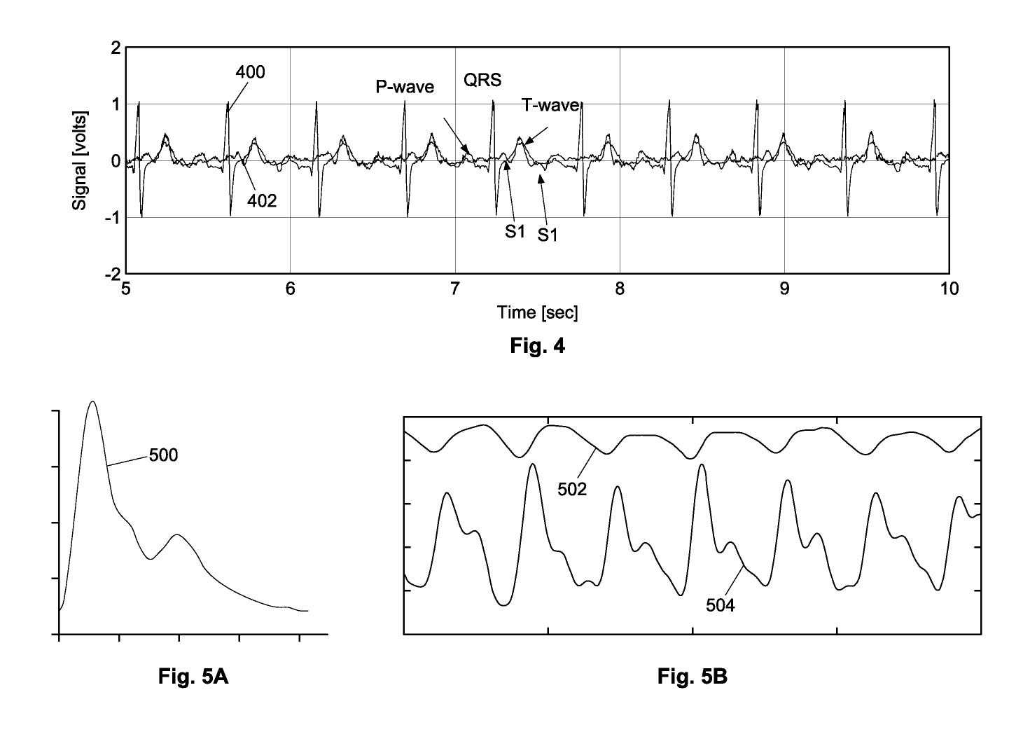

FIG. 5A illustrates a sample of femoral pressure waveform 500. Top curve 502 shows a low-pass filtered CMW signal. Lower curve 504 is the mathematical derivative of the top curve. Comparison shows the similarity of the derivative of the CMW signal (i.e., curve 504) with the femoral pressure waveform 500.

### Embodiment Variations

In addition to the embodiments that been disclosed in detail above, still more are possible within the classes described and the inventors intend these to be encompassed within this specification and claims. The subject disclosure is intended to be exemplary and the claims are intended to cover any modification or alternative which might be predictable to a person having ordinary skill in the art.

Moreover, the various illustrative processes described in connection with the embodiments herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has a user interface port that communicates with a user interface, and which receives commands entered by a user, has at least one memory (e.g., hard drive or other comparable storage, and random access memory) that stores electronic information including a program that operates under control of the processor and with communication via the user interface port, and a video output that produces its output via any kind of video output format, e.g., VGA, DVI, HDMI, DisplayPort, or any other form.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein. The camera may be a digital camera of any type including those using CMOS, CCD or other digital image capture technology.

Likewise, while the subject example is described above as a continuous wave system, a pulsed system is contemplated. Recently available CMOS switches can now provide sub-nanosecond pulses at high microwave frequencies, allowing the transmitter to be turn-off during reception of the return pulse; thus eliminating most leakage correlations. Moreover, using two (or more) frequencies simultaneously between say 1 GHz and 30 GHz (or higher) may allow for the simultaneous measurement of larger heart motions and smaller arterial motions. This type of information would potentially allow for a better diagnosis; and would provide a clearly unique advantage over conventional single-frequency “radar type” systems.

Additionally, improvements to the fine-tuning accuracy and stability of the phase control circuit with the addition of phase-shifters and attenuators with finer tuning ranges in the CW monostatic system may be implemented. Improved algorithms to simultaneously extract features related to large and small physiological related motions, as well as any electrocardiographic-related features may also be used. Such an approach may employ a variety of supervised machine learning techniques (e.g., pre-processing with wavelet transforms to remove gross motion, acyclic dyadic trees with support machine classifiers, auto-segmentation, frequency-domain filters to improve small-feature alignment, etc.). Still further, TEFLON (PTFE lenses or off-axis hyperbolic mirrors can be placed in the beam to focus it down to only a few wavelengths across to target specific organs or veins.

Moreover, the system may be modified to decouple the transmit and receive section of the microwave system and use separate, oppositely circular-polarized antennas for transmit and receive. In which case, circular polarization will change on reflection (from the patient) and it has been shown in active microwave systems to provide >60 dB of leakage isolation. This would practically eliminate any large baseline motion effects, thus simplifying further algorithm development. The remaining motion issues would then likely be due to impedance mismatch at the antennas (and hence a reflection between the two antennas that could lead to a free-space standing wave). However, this issue can readily be addressed with proper antenna design and the use of dual-stub tuners to reduce the mismatch to as low as 50 dB. The reduced gross motion artifacts will be low enough that it will significantly reduce the signal processing requirements to extract the desired features from the reflected CMW signal.

In any case, the steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In one or more exemplary embodiments, the functions described may be implemented in hardware, software, firmware, or any combination thereof. If implemented in software, the functions may be stored on, transmitted over or resulting analysis/calculation data output as one or more instructions, code or other information on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. The memory storage can also be rotating magnetic hard disk drives, optical disk drives, or flash memory based storage drives or other such solid state, magnetic, or optical storage devices. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

Operations as described herein can be carried out on or over a website. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

Also, the inventors intend that only those claims which use the words “means for” are intended to be interpreted under 35 USC 112(f). Moreover, no limitations from the specification are intended to be read into any claims, unless those limitations are expressly included in the claims. The computers described herein may be any kind of computer, either general purpose, or some specific purpose computer such as a workstation. The programs may be written in C, or Java, Brew or any other programming language. The programs may be resident on a storage medium, e.g., magnetic or optical, e.g. the computer hard drive, a removable disk or media such as a memory stick or SD media, or other removable medium. The programs may also be run over a network, for example, with a server or other machine sending signals to the local machine, which allows the local machine to carry out the operations described herein.

Also, it is contemplated that any optional feature of the embodiment variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language. All references cited are incorporated by reference in their entirety. Although the foregoing embodiments been described in detail for purposes of clarity of understanding, it is contemplated that certain modifications may be practiced within the scope of the appended claims.

1. A system for acquiring and analyzing a cardiac microwave (CMW) signal of a subject, the system comprising:
a microwave sensor adapted to capture a signal corresponding the CMW signal; and
at least one computer processor connected to the scanner by a wired or wireless connection, wherein the computer processor is adapted to receive the signal, determine a physiological condition, and output a signal corresponding to the physiological condition.

2. The system of claim 1, wherein the microwave sensor operates in a frequency range of 0.5 GHz to 100 GHz.

3. The system of claim 1, wherein the sensor is adapted to operate in a non-contacting mode.

4. The system of claim 1, wherein the physiological condition corresponds to wall motion of the subject's heart.

5. The system of claim 1, wherein the physiological condition corresponds to micro-motions at the surface of the subject's torso.

6. The system of claim 1, wherein the physiological condition corresponds to wall motion of the subject's aorta.

7. The system of claim 1, wherein the physiological condition corresponds to the subject's blood pressure.

8. The system of claim 1, wherein the physiological condition corresponds to a disease state selected from atherosclerosis, aneurysm, stenosis and hypertension and valvular heart disease.

9. A computer-implemented method of analyzing a signal, comprising:
directing a microwave signal at a subject;
collecting a reflected signal;
analyzing the signal to extract a physiological parameter, the parameter selected from the subject's blood pressure, wall motion of an organ, and a cardiovascular disease state; and
outputting a result of the analyzing.

10. A computer readable medium having stored thereon instructions, which when executed cause one or more processors to:
receive an input signal corresponding to microwave energy reflected from a subject;
analyze the input signal to extract a physiological parameter, the parameter selected from the subject's blood pressure, wall motion of an organ, and a cardiovascular disease state; and
output a result of the extracted parameter.
